(19) United States
(12) Patent Application Publication
(10) Pub. No.: US 2025/0001206 A1
(43) Pub. Date: Jan. 2, 2025

(54) A RADIOACTIVE SOURCE DELIVERY SYSTEMS AND ITS METHOD OF USE

(71) Applicant: HANGZHOU DASHTECH CO., LTD, Hangzhou (CN)

(72) Inventors: Xuetang WANG, Hangzhou (CN); Dingchen ZHU, Hangzhou (CN); Guangming FU, Hangzhou (CN); Xingxing LEI, Hangzhou (CN)

(21) Appl. No.: 18/821,894

(22) Filed: Aug. 30, 2024

Related U.S. Application Data
(63) Continuation of application No. PCT/CN2023/079145, filed on Feb. 28, 2023.

(30) Foreign Application Priority Data
Mar. 3, 2022 (CN) 202210207077.0
Mar. 3, 2022 (CN) 202210208833.1
Aug. 26, 2022 (CN) 202211032981.9
Dec. 9, 2022 (CN) 202211586593.5
Jan. 5, 2023 (CN) 202310014707.7
Jan. 9, 2023 (CN) 202310026341.5

(51) Int. Cl. A61N 5/10

(52) U.S. Cl. CPC A61N 5/1007; A61N 2005/1009; A61N 2005/101; A61N 2005/1011

(57) ABSTRACT

The present invention discloses a radioactive source delivery system and its method of use. The radioactive source delivery system comprise a radioactive source delivery mechanism connecting to the needle trocar via a flexible delivery tube, through which the radioactive source is implanted into a target body; and a needle pulling driver driving the flexible delivery tube which connects to the needle trocar inserted into the target body and controls the needle trocar to be pulled partly out of the target body, thereby adjusting the implantation position of the radioactive source.

This application is a continuation of International Patent Application No. PCT/CN2023/079145, filed on Mar. 1, 2023, the entire contents of which are hereby incorporated by reference.

### TECHNICAL FIELD

The present invention belongs to the technical field of medical devices, specifically relating to a radioactive source delivery system and its method of use in radioactive source implantation surgery.

### BACKGROUND TECHNOLOGY

Radioactive seed implantation surgery involves a direct implantation of multiple radioactive seeds into a tumor through puncture for providing localized radiotherapy. This surgery is applicable to a wide range of cancers, including lung cancer, liver cancer, breast cancer, and prostate cancer. Radioactive seed implantation surgery has small incisions, minimal bleeding, and relatively few complications, yet effectively inhibits tumor growth. Radioactive source implantation surgery has demonstrated its unique therapeutic effects both domestically and internationally. In actual treatment processes, the treatment plans devised based on factors such as the position and size of the tumor target area often require the implantation of multiple seeds, sometimes up to hundreds. Each seed typically needs to be precisely implanted at different locations within the tumor target area, and the puncture angles for each seed can also vary. This results in a time-consuming process for seed implantation. Additionally, during the implantation process, doctors must be in close proximity to the radioactive source, exposing them to significant radiation harm. This severely limits the application and promotion of such surgeries. Therefore, using automated medical devices to replace doctors in performing surgery within a radioactive environment has become an inevitable trend.

Chinese Pub. No. CN1069415A, CN1069063C, CN1190602A, CN1322578A, and CN2235827Y disclose a treatment method and device suitable for various tumors in the human body. Before treatment, a tube or a needle connected with a tube is inserted into the tumor site in the human body, and the radioactive source is fixed to the end of a steel wire rope. It is then delivered through the tube to the tumor site for therapy. After treatment, the steel wire rope and radioactive source are retrieved. In such brachytherapy, the tip of the tube or puncture needle used is sealed (while in the seed implantation surgery, the tip of the hollow puncture needle is open for implanting seeds). And the puncture needle is connected to the front end of a flexible tube, and a radioactive source delivery device is installed at the rear end of the flexible tube. The radioactive source is delivered forward along the tube to the tumor (the radioactive source is not implanted into the body but emits radiation through the tube or the puncture needle). The radiation intensity of this radioactive source is much stronger than that of the I-125 seeds used in seed implantation surgery, achieving radiation therapy effects in just a few minutes. Compared to seed implantation surgery, this kind of therapy has a less irradiate time and cannot suppress tumor growth for an extended period. So, its efficacy in treating certain cancers is not as good as seed implantation surgery. Yet, the seed implantation surgery requires manual intervention to adjust the depth of seed implantation by pulling the needle trocar partly out of the body, which leads to the issue of doctors being exposed to radiation. Additionally, in the seed implantation surgery, the radioactive seeds need to be implanted into the body, so the implantation system need to overcome disinfection and biocompatibility challenges.

Chinese Pub. No. CN110496301A, CN 211214946U, WO2021022971A1 have disclosed a robot for targeted seed implantation in the lithotomy position suitable for clinical use. The robot comprises a frame, a pose adjustment mechanism, a friction wheel seed implantation device with tactile feedback, and a sine elastic amplification torque compensation mechanism. The use of a sine elastic amplification torque compensation mechanism allows for the compensation of gravitational torque in any arbitrary position of the arm, reducing the fluctuation of driving torque and improving the stability of the robot's low-speed operation at the end effector. However, the robot is rigidly connected to the puncture needle, which not only easily scratches the patient during the surgical process, posing a danger, but also leads to a complex and bulky connection structure at the tail end of the puncture needle. It limits the implantation location of radioisotope seeds, and increases the difficulty and duration of the surgical operation.

### Invention Content

To address the aforementioned technical issues, the objective of the present invention is to provide a radioactive source delivery system and its method of use. The puncture needle is flexibly connected to the radioactive source delivery device with a flexible delivery tube, minimizing the risk of patient injury. The needle pulling driver can automatically drive the flexible delivery tube to pull the puncture needle a certain distance from the target body, thereby adjusting the implantation position of the radioactive source. This eliminates the need for manual needle pulling. As the radioactive source is implanted, the needle pulling driver drives the flexible delivery tube to pull the puncture needle partly out of the target body at the same rate and over the same distance, until the radioactive source is fully expelled from the puncture needle. This ensures that the radioactive source is implanted in a stable manner at the target position, improving implantation accuracy and effectiveness. The system achieves fully automated operation, avoids radiation risks, and reduces surgical time.

To achieve the aforementioned objectives, this invention adopts the following technical solutions:

A radioactive source delivery system comprises a radioactive source delivery mechanism, and a needle pulling driver. The needle pulling driver drives the flexible delivery tube which connects to the needle trocar inserted into the target body and controls the needle trocar to be pulled partly out of the target body. The radioactive source delivery mechanism connects to the needle trocar via a flexible delivery tube, through which the radioactive source is implanted into a target body.

Preferably, the radioactive source delivery mechanism comprises a wire output channel, a pushing wire, and a wire driving mechanism. The wire output channel designed to guide the pushing wire to move back and forth within the wire output channel. The wire driving mechanism connects to the wire output channel and is capable of driving the pushing wire to move back and forth along the wire output channel.

Preferably, the radioactive source delivery mechanism also includes a radioactive source feeding mechanism, which is one or a combination of a cutting mechanism, a magazine, and a seed strand feeding mechanism. The radioactive source feeding mechanism is used to set radioactive source at the front of the pushing wire, so that when the wire driving mechanism drives the pushing wire to move forward along the wire output channel, the radioactive source is pushed and delivered to the target body.

Preferably, the radioactive source is seeds or seed strands. The seed strand is a strand containing radioisotope material. The seed strand includes seeds and seed strand casings, the adjacent seeds are either directly abutting each other or separated by the seed strand casings. Alternatively, the seed strand includes seeds and seed strand casings, a plurality of seeds are placed next to each other or at regular intervals within the seed strand casing.

Preferably, the pushing wire is a flexible wire, and the wire driving mechanism is a flexible wire driving mechanism. The flexible wire is a wire with elasticity, which can be bent under the external force and can be restored to a straight state after the external force is removed. The material of the flexible pushing wire is one or a combination of several types, including nickel-titanium alloy, spring steel, and composite materials. The length of the flexible pushing wire is greater than 600 mm.

Preferably, during the implantation procedure, the wire output channel connects to the flexible delivery tube. The wire driving mechanism is capable of driving the pushing wire to move back and forth along the the wire output channel and the flexible delivery tube, so that the radioactive source arranged at the front of the pushing wire by the the radioactive source feeding mechanism is pushed along the flexible delivery tube to the target position.

Preferably, the front end of the flexible delivery tube is equipped with a quick connector designed to be connected with a needle trocar. The quick connector is fixedly connected to the needle trocar using one or a combination of thread, lock mechanism, and adhesive.

Preferably, the flexible delivery tube includes a flexible section. The flexible section is a flexible tube can be bent, with a length exceeding 600 mm, and is made of plastic tube or medical braided tube.

Preferably, the flexible delivery tube comprising:
An inner tube being connected to the needle trocar; and
An outer tube arranged outside the inner tube. One end of the outer tube is pressed against or connected to a support component or the target body. The support component is kept relatively stationary with the target body or mounted on the target body. The inner tube and the outer tube are driven by the needle pulling driver to move relative to each other so that the inner tube pulls the needle trocar partly out of the target body.

Preferably, the support component can be one or a combination of a puncture guidance support arm, a puncture guidance template, a scale-like support component, or a quick-curing support component. The outer tube presses against or connects to the support component, and the needle trocar or inner tube passes through the support component with a clearance.

Preferably, the radioactive source feeding mechanism adopts a magazine which is directly set within the wire output channel. Seeds or seed strands or seed strand casings are loaded into the storage slot or storage hole in the magazine. The magazine feeding mechanism, which is mounted on the magazine, places the seeds or seed strands or seed strand casings at the front end of the pushing wire for supplying. When a seed strand casing is arranged in the magazine, the radioactive source feeding mechanism also includes a seed embedding mechanism, which enables the seed or/and spacer to be embedded into the seed strand casing from one end or the side, forming a complete seed strand.

Preferably, it further comprises a first motion platform and a first connecting portion. One end of the wire output channel and the first connecting portion respectively installed on the opposite sides of the first motion platform, which is used to achieve the relative motion in space of one end of the wire output channel and the first connecting portion. The first connecting portion is one or a combination of an adhesive connecting portion, a welding connecting portion, a threaded connecting portion, a snap-fit connecting portion, or a locking connecting portion.

Preferably, the first connecting portion connects to the connection part which has a plurality of connection holes. The one end of each flexible delivery tube is installed at the corresponding connection hole. The first motion platform is used to achieve the docking between one end of the wire output channel with any flexible flexible delivery tube on the connection part, forming a delivery channel for radioactive source, achieving multi-channel implantation.

Preferably, the first motion platform is one of the following modes:
A. The first connection part moves and one end of the wire output channel remains stationary;
B. The first connection part remains stationary and one end of the wire output channel moves;
C. The first connection part moves and one end of the wire output channel also moves.

Preferably, the first motion platform includes a back-and-forth motion module, a rotation motion module, and a radial motion module. One end of the wire output channel achieves three degrees of freedom of movement in space via the rotational movement in one direction and the linear movements in two directions of the first motion platform.

Alternatively, the first motion platform includes a back-and-forth motion module, a rotation motion module. One end of the wire output channel achieves movement in space via the rotational movement in one direction and the linear movements in one direction of the first motion platform.

Alternatively, the first motion platform includes a back-and-forth motion module, a left-and-right motion module, and an up-and-down motion module. One end of the wire output channel achieves three degrees of freedom of movement in space via the linear movements in three directions of the first motion platform.

Alternatively, the first motion platform is a multi-joint robotic arm which can drive one end of the wire output channel to freely move and position in three-dimensional space. The needle pulling driver drives the inner tube or outer tube of the flexible delivery tube to perform relative sliding motion through direct push and/or pull mechanism, clamp drive mechanism, friction drive mechanism, or meshing drive mechanism.

Preferably, when the direct push and/or pull mechanism is adopted, the needle pulling driver applies a push or pull force directly to the end surface of the inner tube or outer tube, or to the step surface that is set on the inner tube or outer tube. This facilitates the relative sliding motion of the inner tube or outer tube. In such a scenario, the needle pulling driver is a direct push and/or pull mechanism.

Preferably, when the clamp drive mechanism is adopted, a part of the needle pulling driver clamps the inner tube or outer tube, and then this part moves to one side, so as to drive the relative sliding motion of the inner tube or outer tube. In such a scenario, the needle pulling driver is a clamping drive mechanism.

Preferably, when the friction drive mechanism is adopted, a part of the needle pulling driver is pressed against the inner tube or outer tube, and the relative sliding motion of the inner tube or outer tube is driven by the frictional force generated by this part. In such a scenario, the needle pulling driver is a friction drive mechanism.

Preferably, when the meshing drive mechanism is adopted, a gear or a worm of the needle pulling driver engages with the gear groove on the inner tube or outer tube, driving relative sliding between the inner tube or outer tube through the gear or the worm rotation. In such a scenario, the needle pulling driver is a meshing drive mechanism.

Preferably, the needle pulling driver is a direct push and/or pull mechanism. The direct push and/or pull mechanism contains a needle pulling rod and a needle pulling rod driver. The installation direction of the needle pulling rod is in parallel with one end of the wire output channel. The needle pulling rod driver can drive the needle pulling rod to move along the direction that is parallel to the extension direction at the end of the wire output channel. When the front end of the wire output channel is aligned with the inner tube, the needle pulling rod is aligned with the outer tube arranged outside this inner tube. When the needle pulling rod moves forward to push the outer tube, it causes relative sliding between the inner tube and outer tube, pulling out the needle trocar connected at the front end of the inner tube partly out of a target body. The rear end of the outer tube includes a outer tube base, which can be manually adjusted to alter its position relative to the main body of the outer tube, and the adjusted position of the outer tube base can be locked in place through a buckle or threading mechanism.

Preferably, it further comprises a second motion platform and a second connection part. The second motion platform with the direct push and/or pull mechanism and the second connection part respectively installed on the opposite sides, which is used to achieve the relative motion in space of the direct push and/or pull mechanism and the second connection part. The rear end of each flexible delivery tube is connected to a second connection part. The direct push and/or pull mechanism drives any flexible delivery tube on the second connection part by direct pushing-pulling, achieving multi-channel needle pulling.

The second motion platform is one of the following modes:
A. The second connection part moves and the direct push and/or pull mechanism remains stationary;
B. The second connection part remains stationary and the direct push and/or pull mechanism moves;
C. The second connection part moves and the direct push and/or pull mechanism also moves.

Alternatively, the second motion platform is the first motion platform, and the second connection part is the aforementioned connection part. In such a scenario, the direct push and/or pull mechanism and one end of the wire output channel are installed at the same side of the first motion platform. The first motion platform is not only capable of docking the wire output channel with one of the flexible delivery tubes on the connection part for implantation but also can drive the outer tube of this flexible delivery tube through the direct push and/or pull mechanism, pulling out the needle trocar connected to the front end of the flexible delivery tube partly out of the target body.

The use method of a radioactive source delivery system, including the following steps:
a. By setting up the radioactive source delivery mechanism, the needle pulling driver, and the flexible delivery tube. Connecting the front end of the inner tube of each flexible delivery tube to the rear end of a needle trocar that has already been inserted into a target body, and connecting the rear end of the inner tube to the first connecting part. Adjusting the outer tube of the flexible delivery tube so that its front end is pressed against or connected to a support component or the target body.
b. By the movement of the first motion platform, aligning one end of the wire output channel with a flexible delivery tube on the first connecting part, and also aligning the needle pulling rod with the outer tube of this flexible delivery tube. Then, through the back-and-forth motion of the first motion platform, the first motion platform docks one end of the wire output channel with one of the flexible delivery tubes on the first connecting part. The wire driving mechanism drives the pushing wire to move back and forth along the wire output channel, and pushes the radioactive source set in front of the pushing wire forward, through the flexible delivery tube and the needle trocar connected at the front end of the flexible delivery tube, implanting the radioactive source into the target body.

The needle pulling mechanism drives the needle pulling rod to move forward, pushing the outer tube forward. Since the rear end of the inner tube connects and fixes to the connection part, the inner tube and outer tube of the flexible delivery tube slides relative to each other. As the front end of the outer tube of the flexible delivery tube presses against or connects to a support component or the target body, the curvature of the inner tube and outer tube of the flexible delivery tube changes, and the needle trocar retracts relative to the support component or the target body, so as to adjust the depth of the needle front end and control the implantation position of the radioactive source.

Preferably, adopting the synchronized needle pulling and implantation method to achieve the implantation of the radioactive source.

The specific method of synchronized needle pulling and implantation is as follows: when the radioactive source reaches the front end of the needle trocar, the needle pulling driver and the radioactive source delivery mechanism begin to work synchronously. Every time the radioactive source is pushed forward a certain distance, the needle pulling driver drives the flexible delivery tube to pull out the needle trocar by the same distance from the target body at the same rate until the radioactive source is completely pushed out from the needle trocar, so as to implant the radioactive source in a desired posture to the target position.

Preferably, the needle pulling rod of the needle pulling driver passes through the pushing hole on the first connection part to push the outer tube base of the outer tube, so the outer tube of the flexible delivery tube and the inner tube of the flexible delivery tube move relatively to each other. The position of the outer tube base relative to the outer tube is adjust to be near the first connecting portion in advance. This makes the needle trocar to retract a certain distance to control the implantation position of the radioactive source, implanting the radioactive source into the target position. After that, the radioactive source delivery mechanism and the needle pulling driver align with other pushing holes on the first connection part, repeating the above operation to complete the surgery.

### Benefits

The radioactive source delivery system of the present invention can adjust the length and dose of the seed strand according to the characteristics of the tumor and the needs of the surgery, and it also allows for the selection of different types of seed strands. During the surgery, the puncture needle is flexibly connected to the radioactive source delivery device, reducing the risk of patient injury. Additionally, the needle pulling driver mechanism can automatically drive the flexible delivery tube to perform needle pulling, thereby controlling the implantation position of the radioactive source within the target body. Additionally, the radioactive source implantation process and the needle pulling process are synchronized. When the front end of the radioactive source reaches the front end of the puncture needle, the needle pulling driver mechanism and the radioactive source delivery device begin to operate in synchrony. As the radioactive source is implanted for a certain distance, the needle pulling driver mechanism drives the flexible delivery tube to pull the puncture needle from the target object at the same rate and over the same distance, until the radioactive source is completely expelled from the puncture needle. This ensures that the radioactive source is implanted in a stable manner at the target position, improving implantation accuracy and effectiveness. The system achieves fully automated operation, avoids radiation risks, and reduces surgical time.

The present invention enables multi-channel implantation. By setting up the first motion platform and first connection part, one end of multiple flexible delivery tubes is mounted on the first connection part. One end of the wire output channel and the first connection part are mounted on the opposite sides of the first motion platform. The first motion platform is used to achieve relative movement between the end of the wire output channel and the first connection part in space, allowing the wire output channel to dock with each flexible delivery tube on the first connection part to form a delivery channel for the radioactive source delivery. This facilitates multi-channel implantation, offering a simple and efficient structure and drive mechanism.

The present invention enables multi-channel needle pulling. By setting up a second motion platform and second connection part, the direct push-and-pull mechanism and the second connection part are mounted on the opposite sides of the second motion platform. One end of each flexible delivery tube is connected to the second connection part. The second motion platform facilitates relative movement between the direct push-and-pull mechanism and the second connection part in space, allowing the direct push-and-pull mechanism to drive the flexible delivery tube and achieve the pulling of the needle. This enables multi-channel needle pulling. Alternatively, the second motion platform can be the same as the first motion platform, and the second connection part can be the same as the first connection part. By using the first motion platform to achieve relative movement between the direct push-and-pull mechanism and the first connection part in space, a more compact structure is achieved.

### DESCRIPTION OF DRAWINGS

The drawings that form part of this application are provided to offer a further understanding of the application. The illustrative embodiments and descriptions of the application are intended to explain the application and do not constitute limitations on the scope of the application.

FIG. 1 is a structural schematic diagram of Embodiment 1 of the present invention.

FIG. 2 is a schematic diagram of the magazine base, magazine, and the internal structure of the magazine in Embodiment 1 of the present invention.

FIG. 3 is a schematic diagram of Embodiment 1 when the door of equipment housing is opened.

FIG. 4 is a schematic diagram of the internal structure of the drum in Embodiment 1 of the present invention.

FIG. 5 is a schematic diagram of the first connection part in Embodiment 1 of the present invention.

FIG. 6 is a schematic diagram of the synchronized connector locking mechanism in Embodiment 1 of the present invention.

FIG. 7 is a schematic diagram of the needle pulling driver mechanism in Embodiment 1 of the present invention.

FIG. 8 is an enlarged schematic diagram of the area marked L in FIG. 7.

FIG. 9 is a structural schematic diagram of one scheme of the synchronized connector locking mechanism and synchronized stylet locking mechanism in Embodiment 1 of the present invention.

FIG. 10 is a structural schematic diagram of another scheme of the synchronized connector locking mechanism and synchronized stylet locking mechanism in Embodiment 1 of the present invention.

FIG. 11 is a schematic diagram of the seeds being implanted into the target object in Embodiment 1 of the present invention.

FIG. 12 is a schematic diagram of the internal structure of the stylet storage wheel in Embodiment 1 of the present invention.

FIG. 13 is a structural diagram of Embodiment 2 of the present invention.

FIG. 14 is a schematic diagram of the radioactive source delivery device, needle pulling driver, and seed strand feeding mechanism in Embodiment 2 of the present invention.

FIG. 15 is a structural diagram of Embodiment 3 of the present invention.

FIG. 16 is a schematic diagram of the cranial puncture support frame in Embodiment 3 of the present invention.

FIG. 17 is a structural diagram of Embodiment 4 of the present invention.

FIG. 18 is a structural diagram of Embodiment 5 of the present invention.

FIG. 19 is a structural diagram of Embodiment 6 of the present invention.

FIG. 20 is a diagram of the structure at the Y-shaped tube in FIG. 19.

FIG. 21 is a diagram of the structure at the needle pulling rod and the implantation docking tube in FIG. 19.

### DETAILED DESCRIPTION

Further instructions are giver below referring to the drawings and examples, which are not intended to limit the invention.

### Embodiment 1

The present disclosure proposes a radioactive source delivery system comprises a radioactive source delivery mechanism 240, and a needle pulling driver 241 (in this embodiment, the motor B drives the needle pulling rod to move). The needle pulling rod drives the flexible delivery tube which connects to the needle trocar 18 inserted into the target body 1002 and controls the needle trocar 18 to be pulled partly out of the target body. The radioactive source delivery mechanism connects to the needle trocar 18 via a flexible delivery tube, through which the radioactive source is implanted into a target body 1002.

The radioactive source delivery mechanism 240 comprises a wire output channel, a pushing wire, and a wire driving mechanism. The wire output channel designed to guide the pushing wire to move back and forth within the wire output channel. The wire driving mechanism connects to the wire output channel and is capable of driving the pushing wire to move back and forth along the wire output channel.

The radioactive source delivery mechanism also includes a radioactive source feeding mechanism, which is one or a combination of a cutting mechanism, a magazine, and a seed strand feeding mechanism. The radioactive source feeding mechanism is used to set radioactive source at the front of the pushing wire, so that when the wire driving mechanism drives the pushing wire to move forward along the wire output channel, the radioactive source is pushed and delivered to the target body.

The radioactive source is seeds or seed strands. The seed strand is a strand containing radioisotope material. The seed strand includes seeds and seed strand casings, the adjacent seeds are connected by the seed strand casings.

The pushing wire is a flexible wire 24080, and the wire driving mechanism is a flexible wire driving mechanism 24012. The flexible wire is a wire with elasticity, which can be bent under the external force and can be restored to a straight state after the external force is removed. The material of the flexible pushing wire is one or a combination of several types, including nickel-titanium alloy, spring steel, and composite materials. The length of the flexible pushing wire is greater than 600 mm.

During the implantation procedure, the wire output channel connects to the flexible delivery tube 24018. The wire driving mechanism is capable of driving the pushing wire to move back and forth along the the wire output channel and the flexible delivery tube 24018, so that the radioactive source arranged at the front of the pushing wire by the the radioactive source feeding mechanism is pushed along the flexible delivery tube to the target position.

The front end of the flexible delivery tube is equipped with a quick connector 24021 designed to be connected with a needle trocar. The quick connector is fixedly connected to the needle trocar using one or a combination of thread, lock mechanism, and adhesive.

The flexible delivery tube includes a flexible section. The flexible section is a flexible tube can be bent, with a length exceeding 600 mm, and is made of plastic tube or medical braided tube. the flexible delivery tube comprising:
An inner tube being connected to the needle trocar; and
An outer tube arranged outside the inner tube. One end of the outer tube is pressed against or connected to a support component or the target body. The support component is kept relatively stationary with the target body or mounted on the target body 1002. The inner tube or/and the outer tube are driven by the needle pulling driver to move relative to each other so that the inner tube pulls the needle trocar partly out of the target body.

The support component can be one or a combination of a puncture guidance support arm, a puncture guidance template, a scale-like support component, or a quick-curing support component. The outer tube presses against or connects to the support component, and the needle trocar or inner tube passes through the support component with a clearance.

In the present embodiment, the radioactive source feeding mechanism adopts a magazine which is directly set within the wire output channel. Seeds or seed strands or seed strand casings are loaded into the storage slot or storage hole in the magazine. The magazine feeding mechanism, which is mounted on the magazine, places the seeds or seed strand or seed strand casings at the front end of the pushing wire for supplying. When a seed strand casing is arranged in the magazine, the radioactive source feeding mechanism also includes a seed embedding mechanism, which enables the seed or/and spacer to be embedded into the seed strand casing from one end or the side, forming a complete seed strand.

It further comprises a first motion platform (for example, the drum 24010 in this embodiment) and a first connecting portion. The first connecting portion connects to the connection part (for example, the docking plate 2407 in this embodiment). One end of the wire output channel and the first connecting portion respectively installed on the opposite sides of the first motion platform, which is used to achieve the relative motion in space of one end of the wire output channel and the first connecting portion. The first connecting portion is one or a combination of an adhesive connecting portion, a welding connecting portion, a threaded connecting portion, a snap-fit connecting portion, or a locking connecting portion. The connection part has a plurality of connection holes. The one end of each flexible delivery tube is installed at the corresponding connection hole. On the rear end of the flexible delivery tube, there is a quick connection structure (for example, the quick connector 24029 in this embodiment) for connecting the connection hole. The first motion platform is used to achieve the connection between one end of the wire output channel with each flexible delivery tube on the connection part, forming a delivery channel for radioactive source, achieving multi-channel implantation.

Preferably, the first motion platform is one of the following modes:
A. The first connection part moves and one end of the wire output channel remains stationary;
B. The first connection part remains stationary and one end of the wire output channel moves;
C. The first connection part moves and one end of the wire output channel also moves.

The needle pulling driver drives the inner tube or outer tube of the flexible delivery tube to perform relative sliding motion through direct push and/or pull mechanism, clamp drive mechanism, friction drive mechanism, or meshing drive mechanism.

When the direct push and/or pull mechanism is adopted, the needle pulling driver applies a push or pull force directly to the end surface of the inner tube or outer tube, or to the step surface that is set on the inner tube or outer tube. This facilitates the relative sliding motion of the inner tube or outer tube. In such a scenario, the needle pulling driver is a direct push and/or pull mechanism.

When the clamp drive mechanism is adopted, a part of the needle pulling driver clamps the inner tube or outer tube, and then this part moves to one side, so as to drive the relative sliding motion of the inner tube or outer tube. In such a scenario, the needle pulling driver is a clamping drive mechanism.

When the friction drive mechanism is adopted, a part of the needle pulling driver is pressed against the inner tube or outer tube, and the relative sliding motion of the inner tube or outer tube is driven by the frictional force generated by this part. In such a scenario, the needle pulling driver is a friction drive mechanism.

When the meshing drive mechanism is adopted, a gear or a worm of the needle pulling driver engages with the gear groove on the inner tube or outer tube, driving relative sliding between the inner tube or outer tube through the gear or the worm rotation. In such a scenario, the needle pulling driver is a meshing drive mechanism.

The needle pulling driver is a direct push and/or pull mechanism. The direct push and/or pull mechanism contains a needle pulling rod and a needle pulling rod driver. And the installation direction of the needle pulling rod is in parallel with the front end of the wire output channel. The needle pulling rod driver can drive the needle pulling rod to move along the direction that is parallel to the extension direction at the end of the wire output channel. When the wire output channel is aligned with the inner tube, the needle pulling rod is aligned with the outer tube arranged outside this inner tube. When the needle pulling rod moves forward to push the outer tube, it causes relative sliding between the inner tube and outer tube, pulling out the needle trocar connected at the front end of the inner tube partly out of a target body. The rear end of the outer tube includes an outer tube base, which can be manually adjusted to alter its position relative to the main body of the outer tube, and the adjusted position of the outer tube base can be locked in place through a buckle or threading mechanism.

It further comprises a second motion platform and a second connection part. The direct push and/or pull mechanism and the second connection part respectively installed on the opposite sides of the second motion platform, which is used to achieve the relative motion in space of the direct push and/or pull mechanism and the second connection part. Each of the several flexible delivery tubes has an end that is connected to a second connection part. The direct push and/or pull mechanism drives any flexible delivery tube on the second connection part by direct pushing-pulling, achieving multi-channel needle pulling.

The second motion platform is one of the following modes:
A. The second connection part moves and the direct push and/or pull mechanism remains stationary;
B. The second connection part remains stationary and the direct push and/or pull mechanism moves;
C. The second connection part moves and the direct push and/or pull mechanism also moves.

Alternatively, the second motion platform is the first motion platform, and the second connection part is the aforementioned connection part. In such a scenario, the direct push and/or pull mechanism and one end of the wire output channel are installed at the same side of the first motion platform. The first motion platform is not only capable of docking the wire output channel with one of the flexible delivery tubes on the connection part for implantation but also can drive the outer tube of this flexible delivery tube through the direct push and/or pull mechanism, pulling out the needle trocar connected to the front end of the flexible delivery tube partly out of a target body.

In this embodiment, the flexible delivery tube includes an inner tube and an outer tube, the outer tube being sleeved outside the inner tube, the inner tube is clearance-matched with the outer tube and is pluggable relative to the outer tube. The inner tube is configured to be connected with a puncture needle. The inner tube and the outer tube both include a flexible section. The flexible section is made of plastic tube or medical braided tube, and the plastic tube is made of a polytetrafluoroethylene (PTFE) material.

The first motion platform includes a back-and-forth motion module, a rotation motion module. One end of the wire output channel achieves movement in space via the rotational movement in one direction and the linear movements in one direction of the first motion platform.

When the first motion platform is disposed inside a main body housing and includes the back-and-forth movement module and the rotational movement module, a front side of the main body housing is a first connection part panel. The first connection part panel is mounted with a connection part, the connect part is provided with a plurality of connection holes on a rotational track of the first motion platform. Each connection hole may be connected to a flexible delivery tube, and the other end of the flexible delivery tube is connected with a puncture needle. The motion platform is in a shape of a drum, and a wire driving mechanism is disposed in the drum. The rotational movement module drives the drum to rotate around an axis of the drum, and the back-and-forth movement module is disposed in the drum for driving one end of the wire output channel to move forward and backward.

A door is disposed on a left and/or the right side of the main body housing, and a combination of a wire driving mechanism, a stylet pulling mechanism, and a docking tube structure are mounted on the first motion platform by opening the door, or a combination of a wire driving mechanism, a stylet pulling mechanism, a magazine base, and a docking tube structure are mounted on the first motion platform. The stylet pulling mechanism, the magazine base, and the docking tube are mounted perpendicular to the first connection part panel, and a magazine is mounted on the magazine base or mounted on the wire driving mechanism.

When the wire driving mechanism and the magazine base are installed on the first motion platform, the wire driving mechanism and the magazine base are connected by a flexible connection tube, and at this time, the flexible connection tube is a part of the wire output channel, and an outlet of the magazine base is a docking head, which is a terminal end of the wire output channel.

When the wire driving mechanism and the docking tube structure are mounted on the first motion platform, the wire driving mechanism and the docking tube structure are connected by the flexible connection tube, and at this time, the flexible connection tube is a part of the wire output channel, an outlet of the docking tube structure is a terminal end of the wire output channel, and the docking tube structure adopts a floating docking tube structure which can float within a certain range.

The flexible delivery tube is quickly mounted to the connection hole of the first connection part by means of a quick connector, and the first connection part is provided with a synchronized connector locking mechanism, which may lock the quick connectors of all the flexible delivery tubes simultaneously.

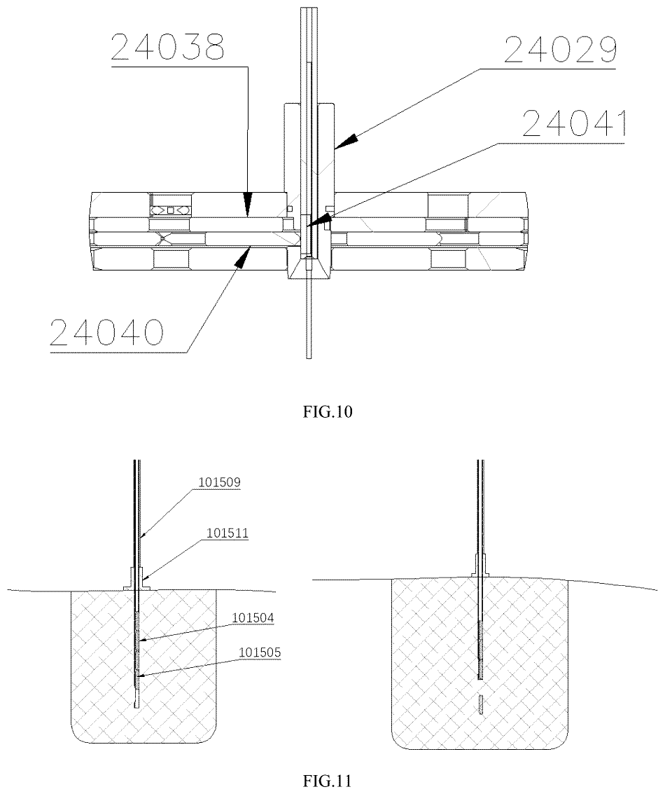

As shown in FIG. 10, the synchronized connector locking mechanism includes a connector clamping plate 24038 disposed within the first connection part, and connector clamping portions are arranged in a circular array on the connector clamping plate 24038. In an unlocked state, all connector clamping portions are staggered with the connection holes, and the quick connector 24029 of the flexible delivery tube can be inserted into the connection holes. When locking, the connector clamping plate 24038 is driven by a clamping driving mechanism to rotate around a center axis, and the connector clamping portions extend into the connection holes. A side of the quick connector 24029 of the flexible delivery tube is provided with a slot, and the quick connector 24029 of the flexible delivery tube is fixed on the first connection part when the connector clamping plate 24038 is rotary to clamp into the slot.

The clamping driving mechanism is electrically or manually actuated and drives the connector clamping plate to rotate around the central axis through cams, gears, or belt drives.

A stylet is disposed inside the flexible delivery tube, extends all the way along the flexible delivery tube, and fills up a space inside the puncture needle connected at a front end of the flexible delivery tube, so as to avoid the blood from pouring into the puncture needle to coagulate and form a clogging. The first connection part is also provided with a synchronized stylet locking mechanism, which may lock the stylet inside all the flexible delivery tubes simultaneously.

As shown in FIG. 10, the synchronized stylet locking mechanism includes a stylet clamping plate 24040 disposed inside the first connection part, stylet clamping portions are arranged in a circular array on the stylet clamping plate 24040. In the unlocked state, all stylet clamping portions are staggered from the connection holes, and the quick connector 24029 of the flexible delivery tube can be inserted into the connection holes. When locking, the stylet clamping plate 24040 rotates around a central axis driven by a stylet driving mechanism, and each of the stylet clamping portions inserts into the interior of each of the connection holes. An opening connected to an elastic body 24041 inside the quick connector 24029 is provided on the side of the quick connector 24029 of the flexible delivery tube, and the elastic body 24041 adopts one or a combination of an elastomer, a rubber tube, or a TPU tube. The elastic body 24041 is adjacent to the stylet, and the stylet clamping plate 24040 presses against the elastic body 24041 during rotation to press down the stylet, thereby realizing the locking.

Alternatively, the synchronized connector locking mechanism is the synchronized stylet locking mechanism, the connector clamping plate is the stylet clamping plate, and the clamping driving mechanism is the stylet driving mechanism, and when the clamping driving mechanism drives the connector clamping plate to rotate around a center axis by a first angle, the connector clamping plate inserts into the slot of the quick connector of the flexible delivery tube while not holding against the elastic body. The connector clamping plate fixes the quick connector of the flexible delivery tube on the first connection part, which just realizes locking of the quick connector of each flexible delivery tube but not realizes locking of the style inside each flexible delivery tube. When the clamping driving mechanism drives the connector clamping plate to rotate around the center axis by a second angle, the connector clamping plate presses against the elastic body during rotation, and then presses the stylet, which realizes locking of each quick connector and the style inside each flexible delivery tube.

The stylet needs to be pulled out before an implantation, the stylet is a flexible wire with elasticity, which may be bent under an external force and restored to a straight state after the external force is withdrawn. A length of the stylet is greater than 600 mm. A rear end of the stylet extends backwardly by a portion from a rear end of the flexible delivery tube, and the rear end of the stylet is provided with a first stopping step, and when the first stopping step is pressed against the rear end of the flexible delivery tube, a distance between a front end of the stylet and the front end of the puncture needle connected to the front end of the flexible delivery tube is no more than 5 mm.

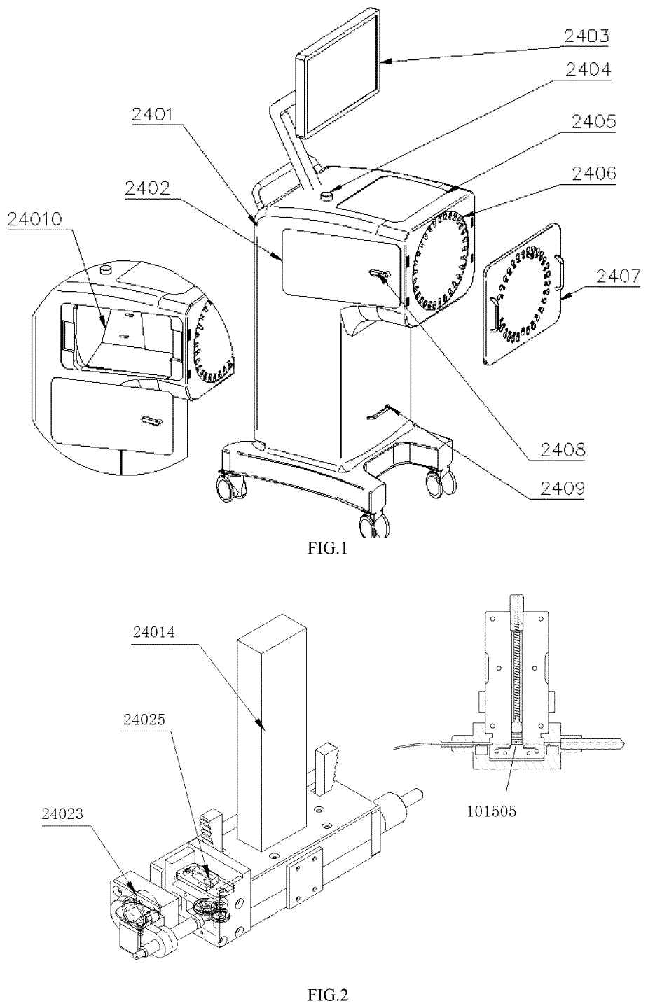

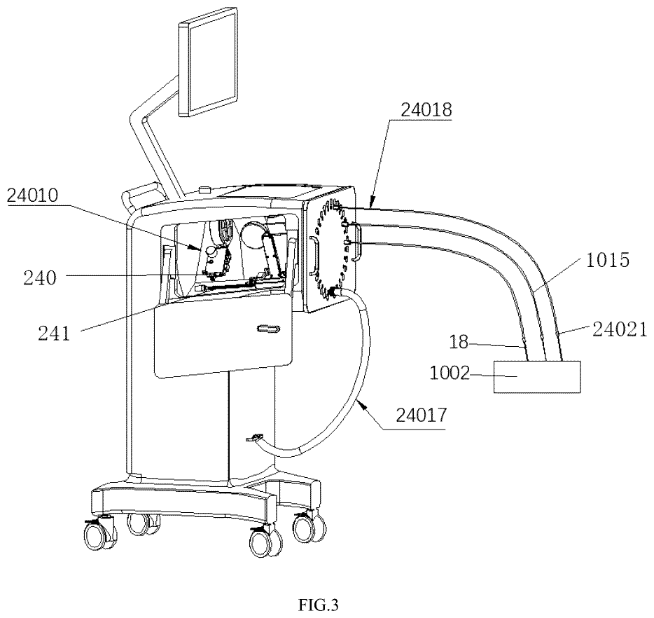

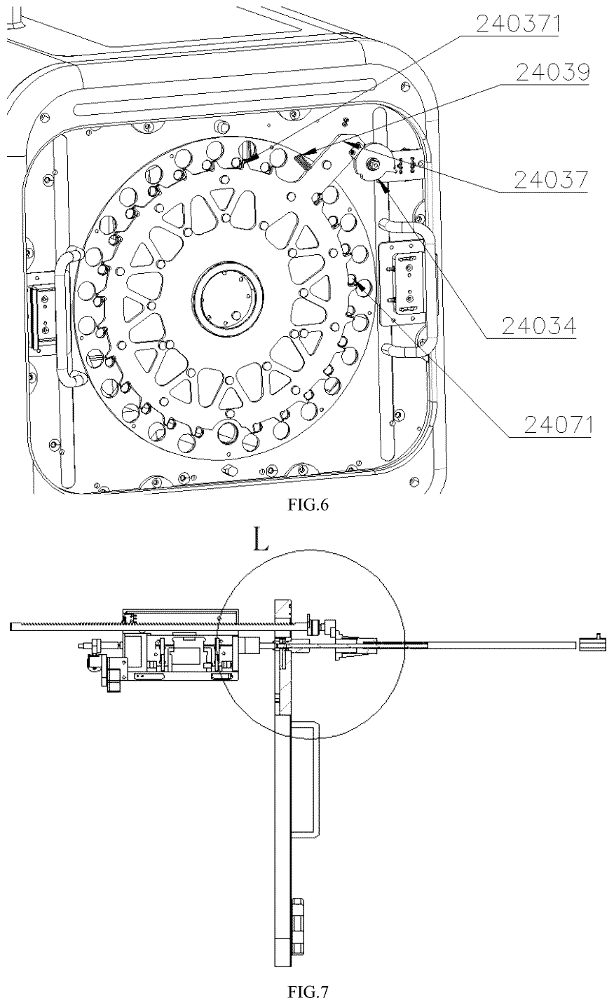

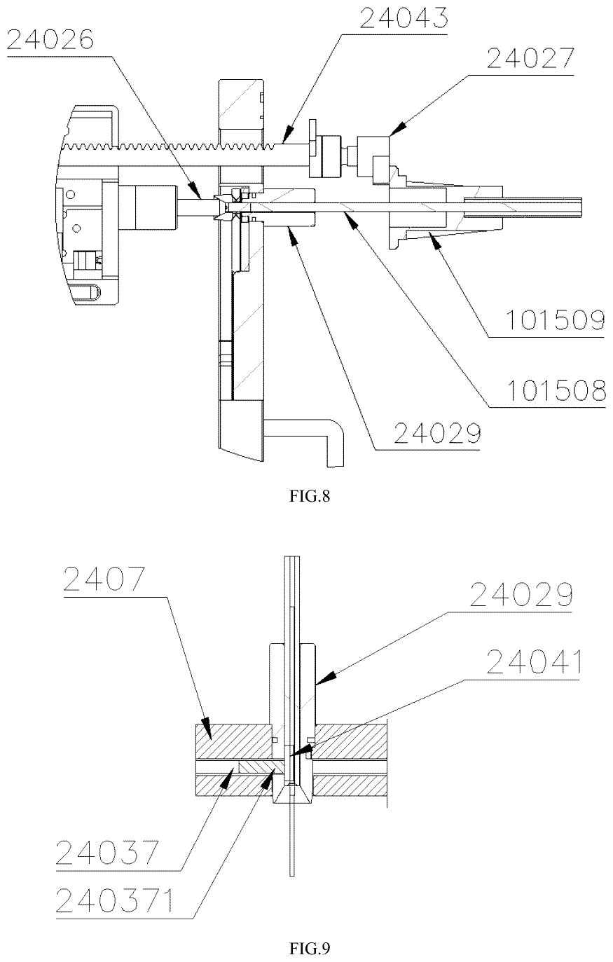

As shown in FIGS. 1-10, the first motion platform is the drum 24010, the drum is disposed inside the main body housing, the drum has a one-direction rotational movement and one-direction linear movement. There is a wire driving mechanism, a stylet pulling docking mechanism and a magazine base inside the drum. The radioactive source feeding mechanism adopts a magazine, the first connection part adopts a docking plate 2407, and a needle pulling driver drives the inner tube or outer tube to make a relative sliding movement between the inner tube or outer tube by direct pushing and pulling.

A main body of a radioactive source implantation mechanism includes a main body housing 2401, a door 2402, a display screen 2403, an emergency stop button 2404, an observation window 2405, a docking plate panel 2406, a docking plate 2407, a door handle 2408, a support column 2409, and a drum 24010.

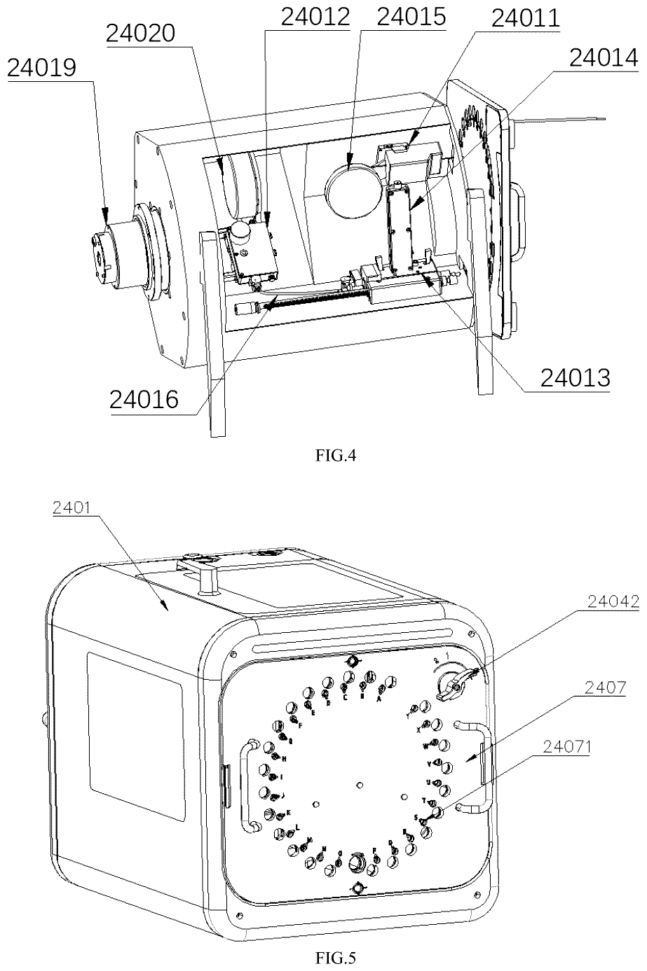

Before the surgery, the door 2402 is in a closed state, the door 2402 is opened by rotate the door handle 2408, the door 2402 is located on the left and/or right side of the main body housing 2401, accessories are installed inside the drum 24010. Specifically, the stylet pulling mechanism 24011, the wire driving mechanism 24012, and the magazine base 24013 are installed in corresponding positions in the drum 24010. The stylet pulling mechanism 24011 and the magazine base 24013 are vertically mounted on the docking plate panel 2406 (referring to FIG. 4). A stylet storage wheel 24015 is disposed at the rear side of the stylet pulling mechanism 24011, and a wire storage wheel 24020 is provided at the rear side of the wire driving mechanism 24012, and the flexible pushing wire 240480 is stored inside the wire storage wheel 24020, and a main material of the flexible pushing wire 24080 is a Ni—Ti alloy wire with a certain degree of toughness. The front end of the Ni—Ti alloy wire is a flexible segment. At the position of the flexible segment, the diameter of the Ni—Ti alloy wire is reduced, and the exterior is filled by the flexible segment, keeping the overall outer diameter of the flexible pushing wire consistent. So, the flexibility of the flexible push wire at the position of the flexible segment and its front end is better. When the flexible pushing wire moves inside the connection tube 24016 and the flexible delivery tube 24018 and encounters a bending position, it has better self-adaptability, which makes it less likely to scratch the delivery tube. At the same time, it can reduce the friction between the flexible pushing wire and the inner wall of the delivery tube. One end of the connection tube 24016 is connected to the wire driving mechanism 24012, the other end of the connection tube 24016 is connected to a force transducer 24023 on the magazine base 24013, and there is a bending section of the connection tube 24016. A magazine 24014 is fitted into the magazine base 24013. The docking plate 2407 (first connection part) is installed on the docking plate panel 2406. A stylet deposit tube 24017 is fixed on a stylet deposit hole of the docking plate 2407. The door 2402 is closed when the installation is complete.

At the beginning of the surgery, a corresponding count of flexible delivery tubes 24018 are prepared in accordance with the count of puncture needles required for the surgery. The quick connectors 24029 of a plurality of flexible delivery tubes 24018 are sequentially installed into connection holes 24071 on the docking plate 2407, a locking handle 24042 is sequentially rotated to a first gear position, a cam 24034 internally connected with the locking handle 24042 rotates to rotate a connector locking plate 24037, the connector locking plate 24037 is provided with a plurality of convex plates 240371 corresponding to the connection holes 24071, and the convex plates 240371 rotate to be embedded in a slot on a side of quick connectors 24029, but not hold against the elastic body 24041, thereby fixing the quick connectors 24029 of the flexible delivery tube 24018 to the docking plate 2407. Then the locking handle 24042 is rotated to a second gear position, the cam 24034 internally connected with the locking handle 24042 rotates to cause the connector locking plate 24037 to continue to rotate, and the convex plate 240371 rotates to press against the elastic body 24041 inside the quick connectors 24429, and the elastic body 24041 simultaneously presses against a stylet inside the flexible delivery tube 24018. Then, the mechanism simultaneously locks all the quick connectors 24429 of the flexible delivery tube 24018 and the stylets inside the flexible delivery tube on the docking plate 2407.

On the other side, an operator holds a puncture needle handle and pierces the puncture needle 18 into a target surgical position of a target body 1002 during the surgery. After the puncture is completed, a needle stylet inside the puncture needle (the needle stylet is very short in length and is used only for puncture) is pulled out, and then the long flexible stylet protruding from the front end of the flexible delivery tube is inserted into the puncture needle to fill the space inside the puncture needle, and then the rear end of the puncture needle is connected to a second quick connector at the front end of the flexible delivery tube, forming a complete implantation channel

Then, turning the locking handle 24042 along an opposite direction to the first gear position, a spring 24039 connected with the connector locking plate 24037 springs back to cause the convex plate 240371 to loosen the elastic body 24041.

When a user starts the seed implantation system, the machine starts to operate, a motor 24019 controls an internal drum 24010 to rotate, thereby making the stylet pulling mechanism 24011 move to align with a flexible delivery tube 24018. Then the stylet pulling mechanism 24011 starts to work and at the same time the stylet pulling docking mechanism drives the stylet pulling mechanism 24011 to move forward to dock with the tail portion of the stylet, and the stylet pulling mechanism 24011 pull out the stylet inside the flexible delivery tube 24018, and the stylet may be delivered to the stylet storage wheel 24015 (a concave structure) on the rear side of the stylet pulling mechanism 24011, the stylet automatically coil inside the stylet storage wheel 24015 under its own elastic action, and drive the stylet storage wheel 24015 to rotate synchronously. After the stylet is completely pulled out of the flexible delivery tube 24018, the stylet pulling mechanism 24011 returns backwardly. The stylet pulling mechanism 24011 is controlled to move back and forth through a linear movement mechanism, and the linear movement mechanism is one or a combination of a screw nut mechanism, an electric pushing rod, and a rack and pinion mechanism. The motor 24019 controls the drum 24010 to rotate so that the stylet pulling mechanism 24011 is aligned with the stylet deposit tube 24017 (aligned with the deposit hole of the docking plate 2407), and the stylet pulling mechanism 24011 then works to completely spit out the stylet and inserts the stylet into the stylet deposit tube 24017.

Subsequently, the motor 24019 drives the drum 24010 to rotate so that the front end of the wire output channel is aligned to the flexible delivery tube 24018 in which the stylet has just been pulled, and the front end of the wire output channel is docked to the quick connector 24029 of this flexible delivery tube 24018. The front end of the wire output channel is controlled to move back and forth through the implantation docking mechanism, and then the wire driving mechanism 24012 pushes out the flexible pushing wire inside the wire storage wheel 24020, and the flexible pushing wire pushes a seed inside the magazine 24014 out of the magazine 24014, and repeats until the front of the magazine 24014 is piled up with a specified number of seeds, and then all of the seeds in front of the flexible pushing wire at once are pushed into the flexible delivery tube 24018 until they are delivered into a second puncture needle 101504.

Subsequently, a needle pulling rod 24043 is pushed forward until it comes into contact with an outer tube base 101509 of outer tube, at which time a contact signal is sensed by a contact sensor 24027, and the contact sensor 24027 is one or a combination of a force sensor, a mechanical switch, an inductive switch, and a photoelectric switch. Then, as shown in FIG. 11, when a plurality of seeds 101505 reach a position of the second puncture needle 101504 connected with the flexible delivery tube, the wire driving mechanism 24012 may push out the first seed into the human body, and then the needle pulling rod 24043 may continue to push the outer tube base 101509 to move forward, and the other end of the outer tube may be pressed against a surface of the body or a puncture template (which may also be an instrument such as a prostate puncture template, etc.), and when the outer tube base 101509 is pushed forward, an inner tube 101508 moves relative to the outer tube and pulls the second puncture needle 101504 connected at the front end of the inner tube partly out of the human body, and then the wire driving mechanism 24012 may continue to push out the seeds 101505 until the plurality of seeds are implanted respectively through repeated operations.

In this scheme, the magazine base 24013 is disposed at the front portion of the wire output channel, a passive measurement wheel is provided inside the magazine base 24013 to press the flexible pushing wire, the friction generated during the movement of the flexible pushing wire drives the passive measurement wheel to rotate, and the rotation angle of the passive measurement wheel is measured by a encoder 24025. A displacement of the flexible pushing wire may be measured by the encoder 24025. Since another set of encoder module and measuring wheel is also disposed inside the wire driving mechanism 24012, then the readings of the two encoders may be used to determine whether the passive measurement wheel is slipping, so as to ensure the reliability of a measurement result based on two measurement results, and through reciprocal movements, seeds being pushed reach a specified count. Then the wire driving mechanism 24012 drives the flexible pushing wire to push out the seeds through the flexible delivery tube 24018 to the front end of the puncture needle. In this process, if a resistance subjected by the flexible pushing wire becomes larger during movement, its reaction force may be transmitted to the force sensor 24023 connected with the connection tube 24016 and fed back.

In another embodiment, reflective stripes or color stripes are provided on the flexible pushing wire, and a photoelectric sensor is disposed in the magazine base 24013 or the wire driving mechanism 24012, so that when the relative displacement of the flexible pushing wire and the photoelectric sensor occurs, the reflective stripes or color stripes may synchronously generate a pulse signal, and an actual displacement of the flexible pushing wire may be measured by sampling the pulse signal, and this solution can avoid slippage between the passive measurement wheel and the flexible pushing wire that may affect the measurement accuracy and reliability of the encoder.

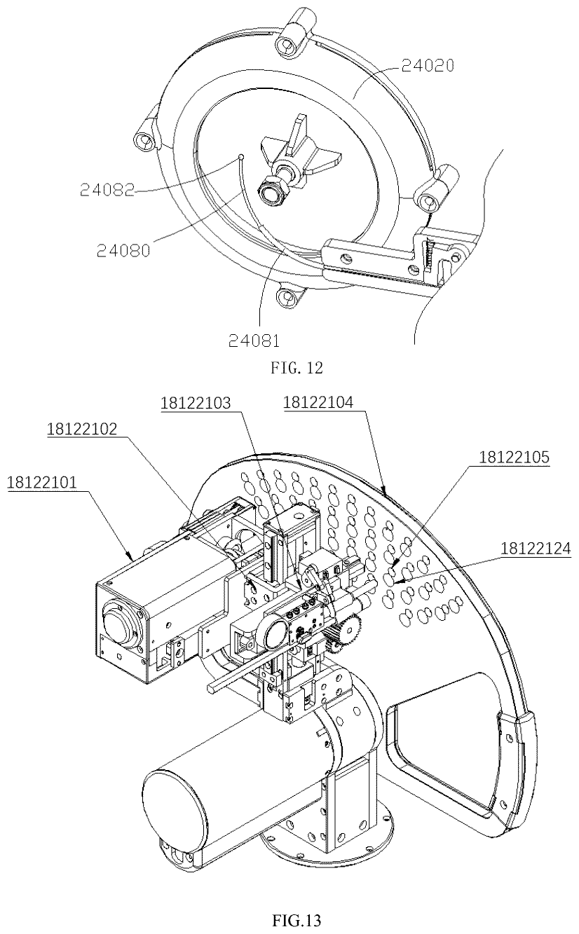

The wire storage wheel 24020 is a wheel-shape container with a concave inner surface (referring to FIG. 12), the wheel-shape container may rotate freely around an axis thereof, and an edge of the wheel-shape container is a concave structure, a storage region is formed at the concave inner surface of the wheel-shape container. At least one side of the wheel-shape container is provided with an opening, and the flexible pushing wire 61 is guided by a storage guiding tube 24081 to extend into the wheel-shape container through the opening on the side of the wheel-shape container. When the wire driving mechanism 24012 drives the flexible pushing wire to move back and forth, under an elastic action of the flexible pushing wire, the flexible pushing wire is automatically coiled inside a concave inner surface of the wire storage wheel 24020, and the wire storage wheel 24020 rotates along with the back-and-forth movement of the flexible pushing wire, thereby realizing automatic storage.

In other embodiments, a second stopping step 24082 may be added to the rear end of the flexible pushing wire, and a position of the second stopping step is set to satisfy following principles: when the front end of the flexible pushing wire extends from the flexible delivery tube into the puncture needle and probes out from the front end of the puncture needle by more than a certain length (the length is less than 10 mm), the second stopping step is stuck on the rear step of the storage guiding tube, so as to realize the mechanical position limiting, which prevents the flexible pushing rod from penetrating further into body tissue, thus ensuring overall safety. Preferably, the second stopping step is an end surface of a stopping tube socketed to the rear end of the flexible pushing wire, or the second stopping step is a welded ball at the rear end of the flexible pushing wire. When the wire driving mechanism 24012 drives the flexible pushing wire to move backward again, the flexible pushing wire is automatically coiled inside the inner concave surface of the wire storage wheel 24020 under the elastic action of the flexible pushing wire, and the wire storage wheel 24020 rotates along with the back and forth movement of the flexible pushing wire, thereby realizing automatic storage.

In other embodiments, a wire connection portion may also be provided on the wire storage wheel 24020, the wire connection portion may be configured to connect and fix the rear end or a middle portion of the flexible pushing wire with the wire storage wheel 24020. A position of the wire connection portion may be set to satisfy following principles: when the front end of the flexible pushing wire extends from the flexible delivery tube into the puncture needle and probes out from the front end of the puncture needle by more than a certain length, the flexible pushing wire is pulled by the wire connection portion so that it is unable to continue to move forward, thereby realizing a mechanical position limiting, which avoids the flexible pushing wire from piercing into the body tissues to cause injuries, and ensuring the overall safety. The wire connection portion adopts a form of threaded connection, bonding, welding, snap connection, and other forms to realize connecting and fixing.

### Embodiment 2

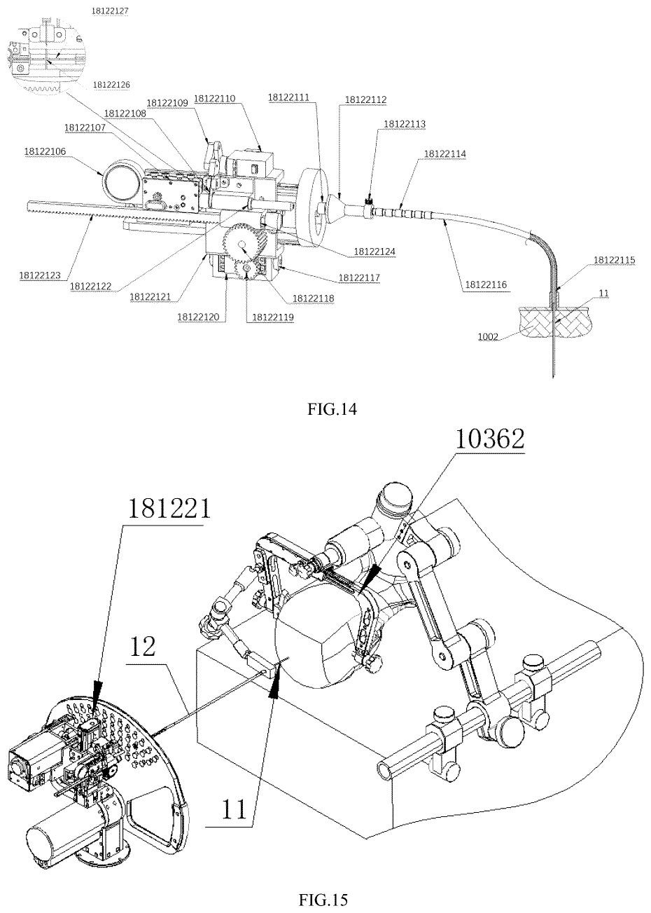

As illustrated in FIGS. 13-14, this embodiment enables automatic switching of the implantation channel. The radioactive source feeding section adopts a cutting mechanism for feeding purposes. In this case, the pushing wire itself serves as the seed strand or seed strand casing, which is then cut by the cutting mechanism to accomplish feeding. The first motion platform functions as the second rotating arm mechanism, and the needle pulling driver drives the needle pulling rod to move back and forth. The needle pulling rod push the outer tube of the needle pulling accessory (flexible delivery tube) to perform relative sliding movement through direct push and/or pull actions.

The working principle is as follows: A friction wheel mechanism 18122107 is provided on the wire driving mechanism 18122103 of the second rotating arm mechanism 18122102. A storage wheel 18122106 is arranged at the end of the friction wheel mechanism 18122107, which is used to store the seed strand 18122127. A docking tube structure 18122122 is arranged at the front end of the friction wheel mechanism, which is fixed on the docking movement base 18122121. A cutting slot is provided at the rear side of the docking tube structure 18122122, and a motor A 18122110 is arranged on the docking movement base 18122121. The motor A 18122110 drives a cutting link mechanism 18122109, which is connected to a cutting blade 18122108. The cutting blade 18122108 is positioned in the cutting slot of the docking tube structure 18122122. A gear rack base 18122124 is arranged below the docking tube structure 18122122, with a gear rack 18122123 set inside the gear rack base 18122124. A motor B 18122120 is arranged at the bottom of the docking movement base 18122121. The side of motor B 18122120 is equipped with or connected to a second force sensor 18122117. The motor B is connected to a driving gear 18122119. A driven gear 18122118 is arranged on the docking movement base 18122121, which meshes with both the driving gear 18122119 and the gear rack 18122123. When the motor B 18122120 drive the gear rack 18122123 and the gear rack 18122123 encounters resistance, the second force sensor 18122117 can detect the reaction force. Motor B is equipped with an angle sensor, allowing for the measuring of the displacement of the gear rack 18122123. Based on force feedback and position feedback, the device can determine whether the gear rack 18122123 has come into contact with the second outer tube base 18122112 or whether the gear rack 18122123 has successfully extended from the second needle pulling holes on the the second connection part 18122104.

Alternatively, the second force sensor 18122117 can be set on the front end of the gear rack 18122123. When the gear rack 18122123 has come into contact with the second outer tube base 18122112, the second force sensor 18122117 generates a force signal as feedback.

An inner tube 18122115 is connected to the second connection part 18122104. The front end of the inner tube 18122115 is connected with an inner tube connector 18122111. A second outer tube 18122116 is provided around the inner tube 18122115, and multiple second metal rings 18122114 are evenly distributed on one end of the second outer tube 18122116. The second outer tube base 18122112 is arranged outside the second metal rings 18122114. The second metal rings 18122114 may also be replaced by locking holes or steps distributed on the second outer tube 18122116. The second outer tube base 18122112 is fixed on the second outer tube 18122116 through a quick locking mechanism.

During the puncture procedure, the inner tube connector 18122111 is secured at the first connection hole 18122105 of the second connection part 18122104. The section of the inner tube 18122115 near the inner tube connector 18122111 is a rigid segment, allowing it to maintain perpendicularity to the second connection part 18122104, thereby providing guidance for the second outer tube base 18122112. The other part of the inner tube 18122115 is a flexible segment, which allows it to flexible connect with puncture needles in various orientations and accommodate the patient's body movements, ensuring surgical safety. Subsequently, the second outer tube base 18122112 is manually moved along the second outer tube 18122116 so that its rear end surface approaches or contacts the second connection part 18122104. The locking screw 18122113 is then adjusted to press against the second metal ring 18122114, securing the second outer tube base 18122112 relative to the second outer tube 18122116. The second metal ring 18122114 is used to prevent the flexible outer tube from being compressed, which could otherwise prevent relative movement between the inner and outer tubes, making needle pulling impossible. Alternatively, the locking screw 18122113 can be inserted into locking holes or steps distributed along the second outer tube 18122116, or replaced with other types of locking mechanisms (e.g., a buckle) to secure the second outer tube base 18122112 relative to the second outer tube 18122116.

The second rotating arm mechanism 18122102 initially aligns the second stylet pulling mechanism 18122101 with the first connection hole 18122105, allowing the second stylet pulling mechanism 18122101 to pull the stylet from the inner tube 18122115 (the stylet is initially set within the inner tube). The second rotating arm mechanism 18122102 then operates to align the docking tube structure 18122122 with the inner tube 18122115. The implantation docking mechanism 18122103 advances the docking tube structure 18122122 to dock with the inner tube 18122115. The friction wheel mechanism 18122107 drives the seed strand 18122127 inside the storage wheel 18122106, where the seed strand 18122127 is primarily composed of seeds and seed strand casings 18122126. After driving the seed strand 18122127 to the target length, motor A 18122110 rotates to drive the cutting link mechanism 18122109, causing the cutting blade 18122108 to rotate and cut the seed strand 18122127 inside the docking tube 18122122. Motor A 18122110 then resets the cutting blade 18122108 to its starting position, and the friction wheel mechanism 18122107 drive the seed strand 18122127 to push the cut seed strand 18122127 through the inner tube 18122115 and the connected puncture needle 11 into the target body.

Simultaneously, motor B 18122120 rotates the driving gear 18122119, driving the driven gear 18122118 to advance the gear rack 18122123. The gear rack 18122123 continues to advance until it contacts the second outer tube base 18122112, at which point the second force sensor 18122117 on the side of motor B 18122120 detects the resistance encountered by motor B 18122120, marking the position of the gear rack 18122123 as the zero point. Motor B 18122120 then continues to rotate, advancing the gear rack 18122123 and pushing the second outer tube base 18122112. The other end of the second outer tube 18122116 has already pressed against the surface of the target body, and the fixed inner tube 18122115 and the pushed second outer tube 18122116 will move relative to each other. As the curvature between the inner and outer tubes changes, the inner tube 18122115 will pull the puncture needle 11 partly out of the target body 1002. While the puncture needle 11 is being withdrawn, the friction wheel mechanism 18122107 simultaneously advances the seed strand 18122127. Then, the seed strand 18122127 pushes the severed seed strand 18122127 into the target position.

In this embodiment, the radioactive source feeding mechanism can also be replaced by the magazine used in Embodiment 1, in which case the implanted object will be the seed rather than the seed strand. However, the principle remains the same and will not be further elaborated. Similarly, the first motion platform in this embodiment can be replaced by the rotating drum structure used in Embodiment 1, as the principle is the same and will not be further elaborated.

### Embodiment 3

This embodiment adopts the radioactive source delivery system from Embodiment 2, which allows for automatic switching of the implantation channel. The radioactive source feeding mechanism uses a cutting mechanism for feeding. In this case, the pushing wire itself is the seed strand or the seed strand casing, which is then cut by the cutting mechanism to achieve feeding. The first motion platform is a rotating arm mechanism, and the needle pulling driver drives the inner or outer tube of the needle pulling component (flexible delivery tube) to slide relatively by direct pushing and/or pulling.

In this embodiment, the support component is a puncture guidance support (such as the cranial puncture support 10362), and the needle pulling driver uses a direct push and/or pull mechanism. A locking mechanism is provided on the support component, which can lock the puncture needle passing through the support component, thus preventing relative movement between the puncture needle and the support component. This ensures that the depth of the puncture needle inserted into the target body is maintained when the user performs other operations. The locking mechanism must be released before the needle pulling driver causes relative sliding between the inner and outer tubes.

The needle pulling driver operates by a direct push and/or pull method and includes a direct push and/or pull mechanism, which consists of a needle pushing part and a needle pushing driver. The needle pushing part and the needle pushing driver are installed parallel to one end of the wire output channel. The needle pushing driver can drive the needle pushing part to move in a direction parallel to the direction of the rear part of the flexible delivery tube. When the wire output channel is docked to the inner tube, the needle pushing part moves forward to push the outer tube, causing relative sliding between the inner and outer tubes, thereby pulling the puncture needle connected to the front end of the inner tube partly from the target body.

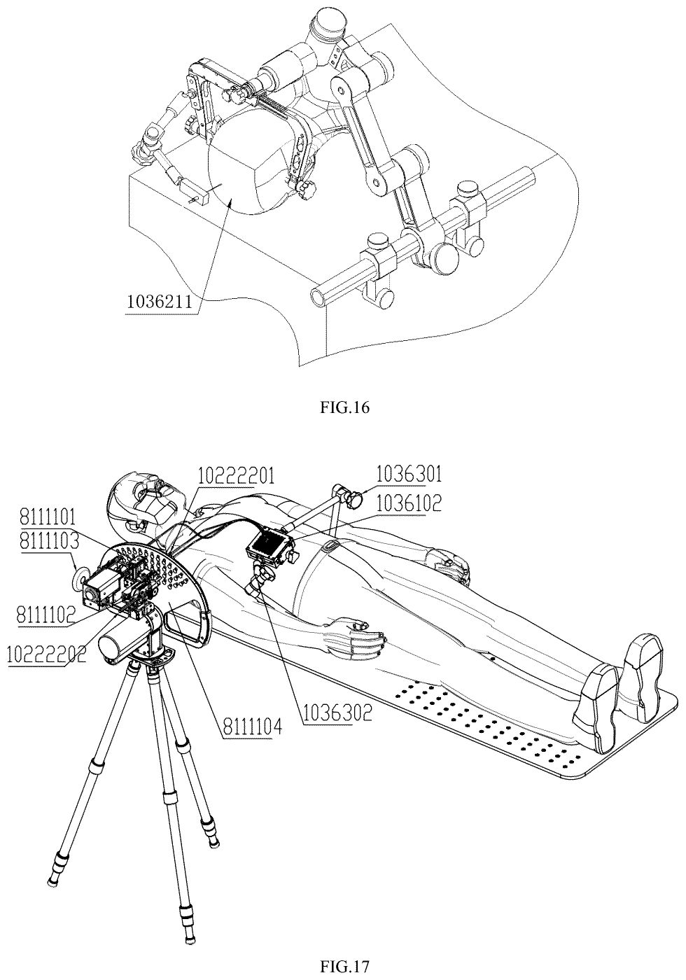

As shown in FIGS. 15-16, this embodiment employs a cranial fixation support, which includes a radioactive source delivery system 181221, a puncture needle 11, a cranial puncture support 10362, and a flexible delivery tube 12. The radioactive source delivery system 181221 is connected to the puncture needle 11 via the flexible delivery tube 12.

The puncture needle 11 (with a short stylet inside) is inserted into the patient's cranium 1036211, and then a CT or MRI scan is performed to verify whether the puncture needle 11 has reached the target location. After confirmation, the short stylet inside the puncture needle 11 is manually pulled out, and one end of the flexible delivery tube 12 is connected to the tail of the hollow puncture needle 11. The other end of the flexible delivery tube 12 is connected to the radioactive source delivery system 181221. Then, the outer tube and the outer tube base (not shown in the figure, similar to Embodiment 2, so not elaborated here) of the flexible delivery tube 12 are adjusted so that the front end of the outer tube is in contact with the cranial puncture support 10362 and the rear end of the outer tube base is near the first connection part.

The radioactive source delivery system 181221 is activated, which implants the seed strand into the patient's cranium 1036211 through the flexible delivery tube 12 and the puncture needle 11. At the same time, the radioactive source delivery system 181221 can achieve relative sliding between the inner tube and the outer tube through pushing the outer tube base, thereby performing the needle pulling action and adjust the implant depth of the puncture needle 11.

In this embodiment, the radioactive source feeding mechanism can also be replaced by the magazine used in Embodiment 1, in which case the implanted object will be the seed rather than the seed strand. However, the principle remains the same and will not be further elaborated. Similarly, the first motion platform in this embodiment (rotating arm mechanism) can be replaced by the rotating drum structure used in Embodiment 1, as the principle is the same and will not be further elaborated.

### Embodiment 4

This embodiment uses the radioactive source delivery system from Embodiment 2, which can automatically switch the implantation channel. The radioactive source feeding mechanism uses the cutting mechanism to supply the seed strand or seed strand casing, which then cuts the seed strand or seed strand casing to achieve feeding.

As shown in FIG. 17, in this embodiment, the support component is a combination of the puncture guidance support arm and the puncture guidance template. The needle pulling driver adopts a direct push and/or pull mechanism, with the adjustable support arm A 1036301 and/or adjustable support arm B 1036302 fixing the array-type puncture guidance template 1036102 in the target position above the patient's skin surface.

Under the guidance of medical imaging such as CT or MRI, the physician inserts the puncture needle through the array-type puncture guidance template 1036102 into the target body. After the puncture needle reaching the target position, the positions of the outer tube and the outer tube base 10222201 of the flexible delivery tube 12 are adjusted so that the front end of the outer tube is in contact with the array-type puncture guidance template 1036102 and the rear end of the outer tube base 10222201 is near the first connection part 8111104.

The swing arm mechanism 8111101 automatically docks the third stylet pulling mechanism 8111103 with a flexible delivery tube on the first connection part 8111104. The first stylet (not shown in the figure) inside this flexible delivery tube is then pulled by the third stylet pulling mechanism 8111103 into the wheeled storage mechanism. Afterward, the docking tube structure is docked with the flexible delivery tube on the first connection part 8111104, and the seed strand implantation device 8111102 cuts the seed strand to the required length and pushes it into the inner tube until it reaches the target position. During the implantation, the needle pulling rod 10222202 passes through the needle pulling hole adjacent to this inner tube on the first connection part 8111104, pushing the outer tube base 10222201, thereby pulling up the corresponding inner tube, i.e., the puncture needle 11, thus adjusting the implantation position of the radioactive source. The above process is repeated for all puncture needles until the procedure is completed.

In this embodiment, the radioactive source feeding mechanism can also be replaced by the magazine used in Embodiment 1, in which case individual seeds, rather than seed strands, are implanted. The principle is the same, so it will not be described again. Similarly, the first motion platform in this embodiment (swing arm mechanism 8111101) can also be replaced by the rotating drum structure used in Embodiment 1, as the principle is the same.

### Embodiment 5

The difference between this embodiment and Embodiment 3 is that this embodiment is used for implanting the seed strand in the prostate area. The support component is a combination of the prostate puncture guidance support frame 62130218 and the array-type puncture guidance template 1036102.

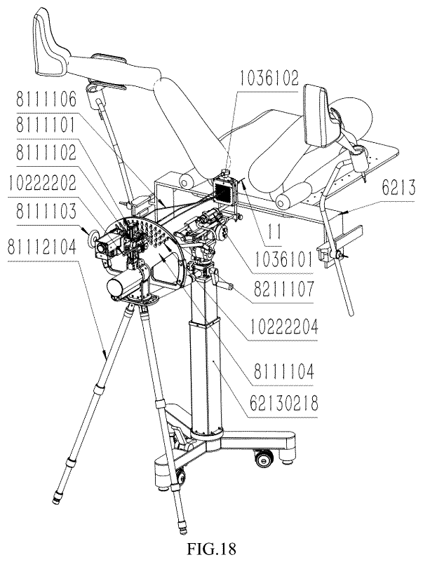

As shown in FIG. 18, the patient is positioned in the lithotomy position on the support of the lithotomy position bracket 6213, with the perineum aligned with the array-type puncture guidance template 1036102. The inner tube (not shown in the figure) is inside the outer tube 8111106, which can slide relative to the inner tube. One end of the inner tube is fixedly connected to the puncture needle 11, while the other end is fixedly connected to the quick connector (not shown in the figure). The implantation quick connector is connected to the connection hole 8211107 on the first connection part 8111104, and one end of the outer tube 8111106 is manually adjust to press against the array-type puncture guidance template 1036102. Under the action of the swing arm mechanism 8111101, the seed strand implantation device 8111102 and the third stylet pulling mechanism 8111103 can dock with each quick connector on the first connection part 8111104. Below each connection hole 8211107 on the first connection part 8111104, there is a corresponding needle pushing hole 10222204, through which the needle pulling rod 10222202 can pass to push the outer tube base, resulting in relative movement between the outer tube and the inner tube, i.e., the puncture needle 11 being pulled up.

Working principle: Adjust the spatial position of the ultrasound probe 1036101 on the prostate puncture guidance support frame 62130218, insert the ultrasound probe 1036101 through the patient's anus, and under the guidance of ultrasound image, insert the puncture needle 11 (with a short stylet inside) through the array-type puncture guidance template 1036102 and the perineum into the prostate. After reaching the target position, the short stylet inside the puncture needle is manually pulled out, and one end of the flexible delivery tube is connected to the tail of the hollow puncture needle. Then, the positions of the outer tube and the outer tube base of the flexible delivery tube are adjusted so that the front end of the outer tube is in contact with the array-type puncture guidance template 1036102 and the rear end of the outer tube base is near the first connection part 8111104.

The swing arm mechanism 8111101 automatically docks the third stylet pulling mechanism 8111103 with a flexible delivery tube on the first connection part 8111104. The first stylet (not shown in the figure) inside this flexible delivery tube is then pulled by the third stylet pulling mechanism 8111103 into the wheeled storage mechanism. Afterward, the docking tube structure is docked with the flexible delivery tube on the first connection part 8111104, and the seed strand implantation device 8111102 cuts the seed strand to the required length and pushes it into the inner tube until it reaches the target position. During the implantation, the needle pulling rod 10222202 passes through the needle pulling hole adjacent to this hole on the first connection part 8111104, pushing the outer tube base 10222201, thereby pulling up the corresponding inner tube, i.e., the puncture needle 11, thus adjusting the implantation position of the radioactive source. The above process is repeated for all puncture needles until the procedure is completed.

In this embodiment, the radioactive source feeding mechanism can also be replaced by the magazine used in Embodiment 1, in which case individual seeds, rather than seed strands, are implanted. The principle is the same, so it will not be described again. Similarly, the first motion platform in this embodiment (the swing arm mechanism 8111101) can also be replaced by the rotating drum structure used in Embodiment 1, as the principle is the same.

### Embodiment 6

In this embodiment, the seed strand implantation device pushes the seed strand, and the Y-shaped tube is used to connect the outlet of the seed strand implantation device 8111102 and the wire output channel 10222103. The needle pulling driver adopts an automatic needle pulling method by pushing the outer tube, synchronizing the automatic needle pulling with the seed strand implantation.

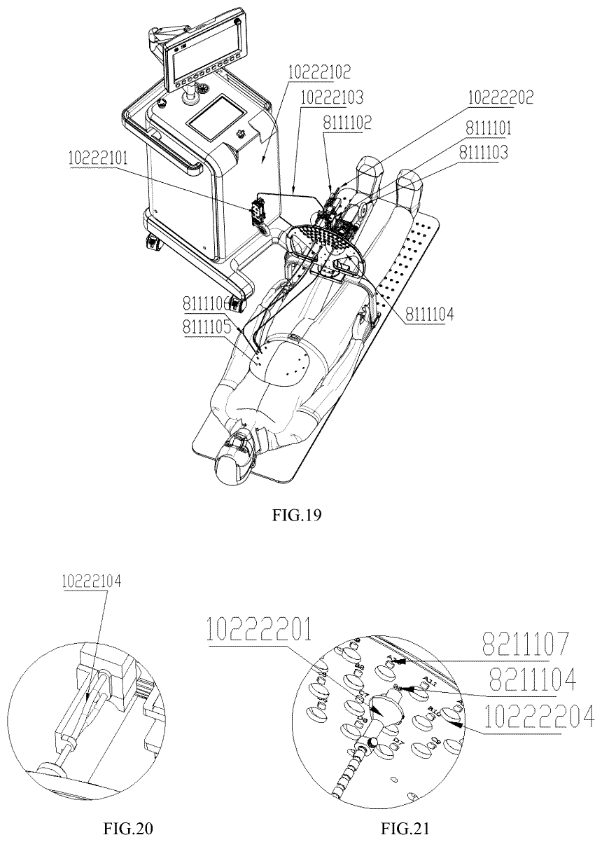

As shown in FIGS. 19-21, in this embodiment, the inner tube (not shown in the figure) is inside the outer tube 8111106 and can slide relative to it. One end of the inner tube is fixedly connected to the puncture needle, while the other end is fixedly connected to the implantation quick connector 8211104. The implantation quick connector 8211104 is connected to the connection hole 8211107 on the first connection part 8111104, and one end of the outer tube 8111106 is pressed against the 3D puncture template 8111105. One end of the wire output channel 10222103 is connected to the wire driving device 10222101, while the other end is connected to one branch of the Y-shaped tube 10222104. The other branch of the Y-shaped tube 10222104 is connected to the outlet of the seed strand implantation device 8111102. The two branches of the Y-shaped tube 10222104 converge into one outlet, which, under the action of the swing arm mechanism 8111101, can dock with each hole on the first connection part 8111104. Below each connection hole 8211107, there is a corresponding needle pulling hole 10222204, through which the needle pulling rod 10222202 can pass to push the outer tube base 10222201, resulting in relative movement between the outer tube and the inner tube, i.e., the puncture needle is pulled up.

After all the puncture needles (with a short stylet inside) are fully inserted into the target body, the short stylet inside the puncture needle is manually pulled out, and one end of the flexible delivery tube is connected to the tail of the hollow puncture needle. Then, the positions of the outer tube and the outer tube base of the flexible delivery tube are adjusted so that the front end of the outer tube is in contact with the 3D puncture template 8111105 and the rear end of the outer tube base is near the first connection part 8111104.

The swing arm mechanism 8111101 automatically docks the third stylet pulling mechanism 8111103 with a flexible delivery tube on the first connection part 8111104. The first stylet (not shown in the figure) inside this flexible delivery tube is then pulled by the third stylet pulling mechanism 8111103 into the wheeled storage mechanism. Afterward, the docking tube structure is docked with this flexible delivery tube on the first connection part 8111104, and the seed strand implantation device 8111102 cuts the seed strand to the required length and pushes it to the front end of the Y-shaped tube 10222104, past the junction of the Y-shaped tube 10222104. The wire driving device 10222101 then drives the flexible pushing wire (not shown in the figure), which passes through the wire output channel 10222103 and the Y-shaped tube 10222104, pushing the seed strand to the target body. During the implantation, the needle pulling rod 10222202 passes through the needle pulling hole adjacent to this hole on the first connection part 8111104, pushing the outer tube base 10222201, thereby pulling up the corresponding inner tube, i.e., the puncture needle 11, thus adjusting the implantation position of the radioactive source. The above process is repeated for all puncture needles until the procedure is completed.

### Embodiment 7

The use method of a radioactive source delivery system, wherein including the following steps:
a. By setting up the radioactive source delivery mechanism, the needle pulling driver, and the flexible delivery tube. Connecting the front end of the inner tube of each flexible delivery tube to the rear end of a needle trocar that has already been inserted into a target body, and connecting the rear end of the inner tube to the first connecting part. Adjusting the outer tube of the flexible delivery tube so that its front end is pressed against or connected to a support component or the target body.
b. By the movement of the first motion platform, aligning one end of the wire output channel with a flexible delivery tube on the first connecting part, and also aligning the needle pulling driver with the outer tube of this flexible delivery tube. Then, through the back-and-forth motion of the first motion platform, the first motion platform docks one end of the wire output channel with one of the flexible delivery tubes on the first connecting part. The wire driving mechanism drives the pushing wire to move back and forth along the wire output channel, and pushes the radioactive source set in front of the pushing wire forward, through the flexible delivery tube and the needle trocar connected at the front end of the flexible delivery tube, implanting the radioactive source into the target body.

The described embodiments are only preferred embodiments of the present invention, and it should be noted that various alterations and improvements may be made therein by those of ordinary skill in the art without departing from the principle of the present invention and should also fall within the scope of protection of the present invention.

Although preferred embodiments have been described herein in detail, it should be noted and will be appreciated by those skilled in the art that numerous variations may be made within the scope of this invention without departing from the principle of this invention and without sacrificing its chief advantages. The terms and expressions have been used herein as terms of description and not terms of limitation. There is no intention to use the terms or expressions to exclude any equivalents of features shown and described or portions thereof and this invention should be defined in accordance with the claims which follow.

We claim:

1. A radioactive source delivery system, comprising:
a radioactive source delivery mechanism connecting to the needle trocar via a flexible delivery tube, through which the radioactive source is implanted into a target body; and
a needle pulling driver driving the flexible delivery tube which connects to the needle trocar inserted into the target body and controls the needle trocar to be pulled partly out of the target body.

2. The radioactive source delivery system according to claim 1, wherein the radioactive source delivery mechanism comprises a wire output channel, a pushing wire, and a wire driving mechanism. The wire output channel designed to guide the pushing wire to move back and forth within the wire output channel. The wire driving mechanism connects to the wire output channel and is capable of driving the pushing wire to move back and forth along the wire output channel.

3. The radioactive source delivery system according to claim 2, wherein the radioactive source delivery mechanism also includes a radioactive source feeding mechanism, which is one or a combination of a cutting mechanism, a magazine, and a seed strand feeding mechanism. The radioactive source feeding mechanism is used to set radioactive source at the front of the pushing wire, so that when the wire driving mechanism drives the pushing wire to move forward along the wire output channel, the radioactive source is pushed and delivered to the target body.

4. The radioactive source delivery system according to claim 1, wherein the radioactive source is seeds or seed strands. The seed strand is a strand containing radioisotope material. The seed strand includes seeds and spacers, the adjacent seeds are either directly abutting each other or separated by the spacer. Alternatively, the seed strand includes seeds and seed strand casing, a plurality of seeds are placed next to each other or at regular intervals within the seed strand casing.

5. The radioactive source delivery system according to claim 1, wherein the pushing wire is a flexible wire, and the wire driving mechanism is a flexible wire driving mechanism. The flexible wire is a wire with elasticity, which can be bent under the external force and can be restored to a straight state after the external force is removed. The material of the flexible pushing wire is one or a combination of several types, including nickel-titanium alloy, spring steel, and composite materials. The length of the flexible pushing wire is greater than 600 mm.

6. The radioactive source delivery system according to claim 2, during the implantation procedure, the wire output channel connects to the flexible delivery tube. The wire driving mechanism is capable of driving the pushing wire to move back and forth along the the wire output channel and the flexible delivery tube, so that the radioactive source arranged at the front of the pushing wire by the the radioactive source feeding mechanism is pushed along the flexible delivery tube to the target position.

7. The radioactive source delivery system according to claim 6, wherein the front end of the flexible delivery tube is equipped with a quick connector designed to be connected with a needle trocar. The quick connector is fixedly connected to the needle trocar using one or a combination of thread, lock mechanism, and adhesive.

8. The radioactive source delivery system according to claim 6, wherein the flexible delivery tube includes a flexible section. The flexible section is a flexible tube can be bent, with a length exceeding 600 mm, and is made of plastic tube or medical braided tube.

9. The radioactive source delivery system according to claim 6, wherein the flexible delivery tube comprising:
An inner tube being connected to the needle trocar; and
An outer tube arranged outside the inner tube. One end of the outer tube is pressed against or connected to a support component or the target body. The support component is kept relatively stationary with the target body or mounted on the target body. The inner tube and the outer tube are driven by the needle pulling driver to move relative to each other so that the inner tube pulls the needle trocar partly out of the target body.

10. The radioactive source delivery system according to claim 9, wherein the support component can be one or a combination of a puncture guidance support arm, a puncture guidance template, a scale-like support component, or a quick-curing support component. The outer tube presses against or connects to the support component, and the needle trocar or inner tube passes through the support component with a clearance.

11. The radioactive source delivery system according to claim 3, wherein the radioactive source feeding mechanism adopts a magazine which is directly set within the wire output channel. Seeds or seed strand or seed strand casings are loaded into the storage slot or storage hole in the magazine. The magazine feeding mechanism, which is mounted on the magazine, places the seeds or seed strand or seed strand casings at the front end of the pushing wire for supplying.
When a seed strand casing is arranged in the magazine, the radioactive source feeding mechanism also includes a seed embedding mechanism, which enables the seed or/and spacer to be embedded into the seed strand casing from one end or the side, forming a complete seed strand.

12. The radioactive source delivery system according to claim 6 further comprising:
a first motion platform. One end of the wire output channel and the first connecting portion respectively installed on the two sides of the first motion platform, which is used to achieve the relative motion in space of one end of the wire output channel and the first connecting portion; and
a first connecting portion being one or a combination of an adhesive connecting portion, a welding connecting portion, a threaded connecting portion, a snap-fit connecting portion, or a locking connecting portion.

13. The radioactive source delivery system according to claim 12, wherein the first connecting portion connects to the first connection part which has a plurality of connection holes. The one end of each flexible delivery tube is installed at the corresponding connection hole. The first motion platform is used to achieve the connection between one end of the wire output channel with any flexible delivery tube on the first connection part, forming a delivery channel for radioactive source, achieving multi-channel implantation.
The first motion platform is one of the following modes:
A. The first connection part moves while one end of the wire output channel remains stationary;
B. The first connection part remains stationary while one end of the wire output channel moves;
C. The first connection part moves while one end of the wire output channel also moves.

14. The radioactive source delivery system according to claim 13, wherein the first motion platform includes a back and forth motion module, a rotation motion module, and a radial motion module. One end of the wire output channel achieves three degrees of freedom of movement in space via the rotational movement in one direction and the linear movements in two directions of the first motion platform.
Alternatively, the first motion platform includes a back and forth motion module, a rotation motion module. One end of the wire output channel achieves movement in space via the rotational movement in one direction and the linear movements in one direction of the first motion platform.
Alternatively, the first motion platform includes a back and forth motion module, a left and right motion module, and an up and down motion module. One end of the wire output channel achieves three degrees of freedom of movement in space via the linear movements in three directions of the first motion platform.
Alternatively, the first motion platform is a multi-joint robotic arm which can drive one end of the wire output channel to freely move and position in three-dimensional space.

15. The radioactive source delivery system according to claim 9, wherein the needle pulling driver drives the inner tube or outer tube of the flexible delivery tube to perform relative sliding motion through direct push and/or pull mechanism, clamp drive mechanism, friction drive mechanism, or meshing drive mechanism.
When the direct push and/or pull mechanism is adopted, the needle pulling driver applies a push or pull force directly to the end surface of the inner tube or outer tube, or to the step surface that is set on the inner tube or outer tube. This facilitates the relative sliding motion of the inner tube or outer tube. In such a scenario, the needle pulling driver is a direct push and/or pull mechanism.
When the clamp drive mechanism is adopted, a part of the needle pulling driver clamps the inner tube or outer tube, and then this part moves to one side, so as to drive the relative sliding motion of the inner tube or outer tube. In such a scenario, the needle pulling driver is a clamping drive mechanism.
When the friction drive mechanism is adopted, a part of the needle pulling driver is pressed against the inner tube or outer tube, and the relative sliding motion of the inner tube or outer tube is driven by the frictional force generated by this part. In such a scenario, the needle pulling driver is a friction drive mechanism.
When the meshing drive mechanism is adopted, a gear or a worm of the needle pulling driver engages with the gear groove on the inner tube or outer tube, driving relative sliding between the inner tube or outer tube through the gear or the worm rotation. In such a scenario, the needle pulling driver is a meshing drive mechanism.

16. The radioactive source delivery system according to claim 15, wherein the needle pulling driver is a direct push and/or pull mechanism. The direct push and/or pull mechanism contains a needle pulling pusher and a needle pulling pusher driver. They are installed in parallel at one end of the wire output channel. The needle pulling pusher driver can drive the needle pulling pusher to move along the direction that is parallel to the extension direction at the end of the wire output channel. When the wire output channel is aligned with the inner tube, the needle pulling pusher is aligned with the outer tube arranged outside this inner tube. When the needle pulling pusher moves forward to push the outer tube, it causes relative sliding between the inner tube and outer tube, pulling out the needle trocar connected at the front end of the inner tube partly out of a target body. The rear end of the outer tube includes a outer tube base, which can be manually adjusted to alter its position relative to the main body of the outer tube, and the adjusted position of the outer tube base can be locked in place through a buckle or threading mechanism.

17. The radioactive source delivery system according to claim 16 further comprising:
a second motion platform with direct push and/or pull mechanism and the second connection part respectively installed on both sides, which is used to achieve the relative motion in space of direct push and/or pull mechanism and the second connection part; and
a second connection part. Each of the several flexible delivery tubes has an end that is connected to a second connection part
The direct push-pull mechanism drives any flexible delivery tube on the second connection part by direct pushing-pulling, achieving multi-channel needle pulling.
The second motion platform is one of the following modes:
A. The second connection part moves while the direct push and/or pull mechanism remains stationary;
B. The second connection part remains stationary while the direct push and/or pull mechanism moves;
C. The second connection part moves while the direct push and/or pull mechanism also moves.
Alternatively, the second motion platform is the first motion platform, and the second connection part is the aforementioned first connection part. In such a scenario, the direct push and/or pull mechanism and one end of the wire output channel are installed at the same side of the first motion platform. The first motion platform is not only capable of docking the wire output channel with one of the flexible delivery tubes on the first connection part for implantation but also can drive the outer tube of this flexible delivery tube through the direct push and/or pull mechanism, pulling out the needle trocar connected to the front end of the flexible delivery tube partly out of a target body.

18. The use method of a radioactive source delivery system, wherein including the following steps:
a. By setting up the radioactive source delivery mechanism, the needle pulling driver, and the flexible delivery tube. Connecting the front end of the inner tube of each flexible delivery tube to the rear end of a needle trocar that has already been inserted into a target body, and connecting the rear end of the inner tube to the first connecting portion. Adjusting the outer tube of the flexible delivery tube so that its front end is pressed against or connected to a support component or the target body.
b. By the movement of the first motion platform, aligning one end of the wire output channel with a flexible delivery tube on the first connecting portion, and also aligning the needle pulling driver with the outer tube of this flexible delivery tube. Then, through the back and forth motion of the first motion platform, the the first motion platform docks one end of the wire output channel with one of the flexible delivery tubes on the first connecting portion. The wire driving mechanism drives the pushing wire to move back and forth along the wire output channel, and pushes the radioactive source set in front of the pushing wire forward, through the flexible delivery tube and the needle trocar connected at the front end of the flexible delivery tube, implanting the radioactive source into the target body.
The needle pulling mechanism drives the needle pulling pusher to move forward, pushing the outer tube forward. Since the rear end of the inner tube connects and fixes to the first connection part, the inner tube and outer tube of the flexible delivery tube slides relative to each other. As the front end of the outer tube of the flexible delivery tube presses against or connects to a support component or the target body, the curvature of the inner tube and outer tube of the flexible delivery tube changes, and the needle trocar retracts relative to the support component or the target body, so as to adjust the depth of the needle front end and control the implantation position of the radioactive source.

19. The use method of a radioactive source delivery system according to claim 18, wherein adopting the synchronized needle pulling and implantation method to achieve the implantation of the radioactive source.
The specific method of synchronized needle pulling and implantation is as follows: When the radioactive source reaches the front end of the needle trocar, the needle pulling driver and the radioactive source delivery mechanism begin to work synchronously. Every time the radioactive source is pushed forward a certain distance, the needle pulling driver drives the flexible delivery tube to pull out the needle trocar by the same distance from the target body at the same rate until the radioactive source is completely pushed out from the needle trocar, so as to implant the radioactive source in a standard posture to the target position.

20. The use method of a radioactive source delivery system according to claim 18, wherein the needle pulling pusher of the needle pulling driver passes through the pushing hole on the first connection part to push the pusher base on the outer tube, so the outer tube of the flexible delivery tube and the inner tube of the flexible delivery tube move relatively to each other. The position of pusher base relative to the outer tube is adjust to be near the first connecting portion in advance. This makes the needle trocar to retract a certain distance to control the implantation position of the radioactive source, implanting the radioactive source into the target position. After that, the radioactive source delivery mechanism and the needle pulling driver align with other pushing holes on the first connection part, repeating the above operation to complete the surgery.
